(19) United States
(12) Patent Application Publication
(10) Pub. No.: US 2024/0279614 A1
(43) Pub. Date: Aug. 22, 2024

(54) METHOD FOR CRYOPRESERVATION AND RESUSCITATION OF FOLLICLES

(71) Applicant: University of Science and Technology of China, Hefei (CN)

(72) Inventors: Gang ZHAO, Hefei (CN); Conghui TIAN, Hefei (CN); Lingxiao SHEN, Nanjing (CN)

(21) Appl. No.: 18/652,960

(22) Filed: May 2, 2024

Related U.S. Application Data
(63) Continuation of application No. PCT/CN2023/081808, filed on Mar. 16, 2023.

(30) Foreign Application Priority Data
Mar. 29, 2022 (CN) 202210318082.9

(51) Int. Cl. C12N 5/071; A01N 1/02

(52) U.S. Cl. CPC C12N 5/0682; A01N 1/0221; A01N 1/0231; A01N 1/0284; C12N 2500/25; C12N 2501/11; C12N 2501/235; C12N 2501/31; C12N 2529/00; C12N 2531/00

(57) ABSTRACT

Methods for cryopreservation and resuscitation of follicles are provided by the present disclosure, relating to the technical field of follicular cryopreservation. According to the method for cryopreserving follicles provided by the present disclosure, follicles are firstly encapsulated with Alginate hydrogel, and incubated in protectant A and B. and moved into a protective solution A for incubation. Follicles encapsulated in hydrogel microcapsules are then loaded into straws and immediately submerged into liquid nitrogen for cryopreservation. Then, they are rapidly warmed by a 37° C. water bath combined with nano-warming technique. To verify the preservation effect, the warmed preantral follicles are further cultured in vitro in three dimensions.

### CROSS-REFERENCE TO RELATED APPLICATIONS

This application is a continuation of PCT/CN2023/081808, filed on Mar. 16, 2023, and claims priority of Chinese Patent Application No. 202210318082.9, filed on Mar. 29, 2022, the entire contents of which are incorporated herein by reference.

### TECHNICAL FIELD

The present disclosure relates to the technical field of follicular cryopreservation, and in particular to methods for cryopreservation and resuscitation of follicles.

### BACKGROUND

Recent years have seen increasing incidences of some malignant diseases (including breast cancer, haematological diseases) among young women. Although methods of intensive chemotherapy, radiotherapy or bone marrow transplantation provide a cure of 90 percent of girls and young women, such treatments nevertheless impair gonadal function and may even lead to premature ovarian failure. As a result, the requirement for female fertility preservation has increased dramatically. For girls before puberty with certain specific diseases (e.g. acute leukaemia, ovarian cancer) and women in urgent need of cancer treatment, cryopreservation of preantral follicles is one of the reliable options.

The development of follicles is generally divided into stages of primordial follicles, primary follicles, secondary (or preantral) follicles, tertiary (or antral) follicles and mature follicles. Among them, preantral follicles which exist in the gonads of individuals of all ages and can also be obtained from the ovaries of sexually mature women.

The commonly cryopreserved follicles are sinus follicles, with methods mainly including slow cryopreservation and vitrification cryopreservation, and it is considered that vitrification cryopreservation is a more effective method of cryopreservation because of the simplicity of the equipment required and the ease of handling compared to traditional slow cryopreservation. Vitrification cryopreservation usually involves using high concentrations of cryoprotectants to bring the cells into an amorphous vitrified state at ultra-low temperatures. However, high concentrations of osmotic cryoprotectants (up to 6-10 mole per liter) may induce uncontrollable osmotic damage and biotoxicity, severely affecting the efficacy of the preservation and the subsequent development of the follicles. Moreover, the traditional method of re-warming is to place the cryopreserved tubes in a 37 degrees Celsius thermostatic water bath and passively re-warm them while shaking. Such a method is not efficient enough in warming and leads to severe anti-vitrification and recrystallisation, which can be fatal for preantral follicles.

Therefore, methods for cryopreservation of preantral follicles with low concentration of protectants for freezing as well as rapid warming is urgently required.

### SUMMARY

In view of this, a technical problem to be solved by the present disclosure is to provide methods for cryopreservation and resuscitation of follicles using protectants for freezing at low concentrations as well as rapid and effective warming.

The present disclosure provides a method for cryopreservation of follicles, including:
encapsulating follicles with sodium trehalose gel, followed by moving into a protective solution A for incubation, then moving into a protective solution B for another incubation, and immersing in liquid nitrogen for cryopreservation;
the protective solution A includes cryoprotectants;
the protective solution B includes cryoprotectants, Fe₃O₄ nanoparticles and graphene oxide (GO) nanoparticles; and
the cryoprotectants are selected from one or a combination of two or more of dimethyl sulfoxide, glycerol, ethylene glycol, propylene glycol, trehalose, acetamide or methanol.

In the present disclosure, the cryoprotectants include ethylene glycol, propylene glycol and trehalose.

In the present disclosure, a diameter of the Fe₃O₄ nanoparticles is in a range of 10-50 micrometer (μm), and a diameter of the GO nanoparticles is in a range of 200-400 μm.

In some specific embodiments, the diameter of the Fe₃O₄ nanoparticles is 20 μm, and the diameter of the GO nanoparticles is 300 μm.

According to the method for cryopreservation, the follicles are firstly encapsulated by hydrogel, where the hydrogel microencapsulation is used as a protective barrier to separate preantral follicles from nanoparticles, so as to effectively inhibit the toxicity of nanoparticles; moreover, hydrogel microencapsulation is capable of effectively preventing the inward growth of ice crystals, therefore reducing the damage of extracellular ice and the concentration of osmotic protectant; in addition, the three-dimensional network structure of hydrogel is similar to extracellular matrix, which can be used for in vitro culture of preantral follicles after resuscitation; however, low concentration of sodium alginate is selected for the preparation of hydrogel as the hardness and concentration of hydrogel affects the in vitro development of preantral follicles.

According to the method for cryopreservation, the encapsulating follicles with sodium trehalose gel includes: mixing follicles with 0.5 weight percentage (wt %)—2 wt % sodium trehalose solution, followed by drop-adding 0.1 mole per liter (mol/L)—0.5 mol/L calcium chloride solution.

In the present disclosure, a step of the encapsulating includes performing encapsulating using a centrifugal tube with a nozzle. A volume of the centrifugal tube is 1.5 milliliters (mL), the nozzle is configured on a top of the centrifugal tube, and an inner diameter of the nozzle is in a range of 100-500 μm. The nozzle of the centrifugal tube is filled with sodium trehalose solution of 0.5 wt %-2 wt %, and a bottom of the centrifugal tube is filled with calcium chloride solution of 0.1 mol/L-0.5 mol/L. Steps of performing encapsulating include: adding follicles into the nozzle filled with sodium trehalose solution, and centrifuging to make the sodium trehalose solution in the nozzle and follicles fall into the calcium chloride solution at the bottom of the centrifugal tube under an action of a centrifugal force to form hydrogel microspheres. Under concentrations of sodium trehalose and calcium chloride as well as the inner diameter of the nozzle illustrated above, the centrifugal force of 100×g-500×g is more stable for preparing the microspheres.

According to the method for cryopreservation, the hydrogel microspheres encapsulated with follicles are moved to a straw containing a protectant A for incubation for 20 minutes (min), and then a protectant B is added for incubation for 10 min.

According to the method for cryopreservation, the protective solution A includes water and 0.5 mol/L-2 mol/L ethylene glycol, 0.5 mol/L-2 mol/L propylene glycol and 0.5 mol/L-2 mol/L trehalose. In some embodiments, the protective solution A includes water and 1 mol/L of ethylene glycol, 1 mol/L of propylene glycol and 1 mol/L of trehalose.

According to the method for cryopreservation, the protective solution B includes water and 0.5 mol/L-2 mol/L ethylene glycol, 0.5 mol/L-2 mol/L propylene glycol, 0.5 mol/L-2 mol/L trehalose, 0.1 wt %-1 wt % Fe₃O₄ nanoparticles and 0.01 wt %-0.1 wt % GO nanoparticles. In some embodiments, the protective solution B includes water and 1 mol/L ethylene glycol, 1 mol/L propylene glycol, 1 mol/L trehalose, 0.5 wt % Fe₃O₄ nanoparticles and 0.05 wt % GO nanoparticles.

The protectant B of the present disclosure is frozen together with follicles, and contains two kinds of nanoparticles with magnetocaloric (Fe₃O₄) and photothermal properties (GO) respectively. The two nanoparticles can be adjusted at will in terms of concentration and ratio to obtain a suitable heating rate. On a basis of constant temperature water bath at 37 degrees Celsius (° C.), the vitrification preservation using low permeability protectant (1-3 mol/L) for preantral follicles is realized by nano-reheating combined with magnetic induction heating and light induction heating. In a process of resuscitation, Fe₃O₄ nanoparticles and GO nanoparticles contained in protectant B realize rapid and uniform warming under a combined action of magnetic induction heating and light induction heating, thus improving a survival rate of the follicles.

According to the present disclosure, the follicles are preantral follicles. Preantral follicle is the early stage of follicle and accounts for a large proportion in ovarian cortex and has great potential in preservation, reproduction or research.

A method for resuscitating frozen follicles obtained by the method for cryopreservation includes: placing the frozen follicles in a water bath of 37° C., and carrying out magnetic induction heating and near infrared heating.

According to the present disclosure, the frozen follicles are loaded in the straw, and the straw is placed in a 37° C. water bath.

A current of the magnetic induction heating is 5 amperes (A) to 30 A, preferably 15 A. A power of the near infrared heating is 1 watt per square centimeter (W/cm²) to 10 W/cm², preferably 3 W/cm².

An in vitro culture method for obtaining follicles of the method for resuscitating frozen follicles includes: culturing resuscitated cells in a culture medium; the culture medium includes a culture medium A and a culture medium B. The culture medium A and the culture medium B provided by the present disclosure have significant effects on culture results, and similar culture effects cannot be obtained under other culture medium conditions.

According to the present disclosure, the culture medium A includes: α-minimum essential medium (α-MEM), 8% (v/v)-12% (v/v) fetal bovine serum (FBS), 80-120 milli-international units per milliliter (mIU/mL) follicle-stimulating hormone (FSH), 1×-2× insulin-transferrin-selenium (ITS), 800-1200 IU/mL leukemia Inhibitory Factor (LIF) and 3-7 micrograms per milliliter (μg/mL) epidermal growth factor (EGF). In some embodiments, the culture medium A includes: α-MEM, 10% (v/v) FBS, 100 mIU/mL FSH, 1×ITS, 1000 IU/mL LIF and 5 μg/mL EGF.

According to the present disclosure, the culture medium B includes: α-MEM, 8% (v/v)-12% (v/v) FBS, 80-120 mIU/mL FSH+1×-2×ITS+800-1200 IU/mL LIF+3-7 μg/mL EGF, 150-250 mIU/mL luteinizing hormone (LH) and 2-3 U/mL human chorionic gonadotropin (HCG). In some embodiments, the culture medium B includes: α-MEM, 10% (v/v) FBS, 100 mIU/mL FSH+1×ITS+1000 IU/mL LIF+5 μg/mL EGF, 200 mIU/mL LH and 2.5 U/mL HCG.

According to the present disclosure, an intensity of the current of electromagnetic induction and the power of infrared heating are required to be strictly matched with an addition amount of the nanoparticles in the protectant B. The relevant parameters documented in the specification of the present disclosure are all obtained after extensive screening work, for instance, adjustments to the contents of Fe₃O₄ nanoparticles and GO nanoparticles are combined with other electromagnetic intensities or infrared power, all of which may have an impact on the resuscitation effect and ultimately lead to a lower survival rate of less than 90% of the follicles.

According to the present disclosure, the resuscitated follicles are also encapsulated with hydrogel, so the resuscitated preantral follicles are capable of being directly cultured in-situ in vitro in the hydrogel without any treatment, and a good culture effect is obtained.

According to the method for cryopreservation provided by the present disclosure, follicles are encapsulated firstly so as to effectively prevent the inward growth of ice crystals and reduce extracellular ice damage and the concentration of osmotic protectants; under the isolation of hydrogel, no toxicity is produced to the follicles as adding the nanoparticles into the frozen solution, and the nanoparticles are capable of heating the frozen follicles evenly by magnetic induction heating and light induction heating, thus improving the resuscitation efficiency and the survival rate of the follicles after resuscitation. Moreover, the three-dimensional network structure of encapsulated hydrogel is similar to extracellular matrix, which can be used for in vitro culture of preantral follicles after resuscitation.

### BRIEF DESCRIPTION OF THE DRAWINGS

FIG. 1 illustrates a process of hydrogel microencapsulation of preantral follicles.

FIG. 2 shows processes of incubation of protectant A and the addition of nanoparticles, vitrification cryopreservation of preantral follicles, and nano-warming of preantral follicles.

FIG. 3 shows the in-situ culture of preantral follicles after warming.

### DETAILED DESCRIPTION OF THE EMBODIMENTS

The present disclosure provides methods for cryopreservation and resuscitation of follicles. A person skilled in the art may draw on the contents herein and improve the process parameters to achieve as appropriate. It is particularly noted that all similar substitutions and modifications are apparent to a person skilled in the art, and are considered to be included in the present disclosure. The methods and applications of the present disclosure are described by means of preferred embodiments, and it is obvious that the person concerned is capable of realizing and applying the technology of the present disclosure by making alterations or appropriate changes and combinations to the methods and applications herein without departing from the content, spirit and scope of the present disclosure.

This disclosure combines hydrogel microencapsulation and nano-warming technology to realize the cryopreservation of preantral follicles. To verify the preservation effect, the warmed preantral follicles are further cultured in vitro in three dimensions. According to the methods, the preantral follicles are microencapsulated with hydrogel by a centrifugal device, which is simple, convenient and effective to prevent the loss of preantral follicles during encapsulation. On the basis of constant temperature water bath at 37 degrees Celsius (° C.), nano-warming combined with magnetic induction heating and light induction heating is used to heat preantral follicles, realizing rapid and uniform warming, thus effectively shorten the duration of devitrification or recrystallization, and reducing the required osmotic protectants used in vitrification cryopreservation. The preantral follicles and nanoparticles are completely separated using hydrogel microencapsulation, and the potential toxicity of nanoparticles to preantral follicles are therefore effectively inhibited; moreover, hydrogel microencapsulation prevents ice crystals from growing into cells during freezing, further contributing to the reducing of the concentration of osmotic protectants from another perspective. In addition, the three-dimensional network structure of hydrogel is similar to extracellular matrix, which can be used for in vitro culture of preantral follicles. The present disclosure successfully realizes vitrification cryopreservation and in-situ culture after cryopreservation of mouse preantral follicles (in hydrogel microcapsules previously used for cryopreservation). By adopting the method, mature oocytes and healthy mice are obtained, providing technical support for the preservation of female fertility and related research.

The samples used in the present disclosure are all common commercial products, which can be purchased in the market. The present disclosure is further illustrated with embodiments.

### Embodiment 1

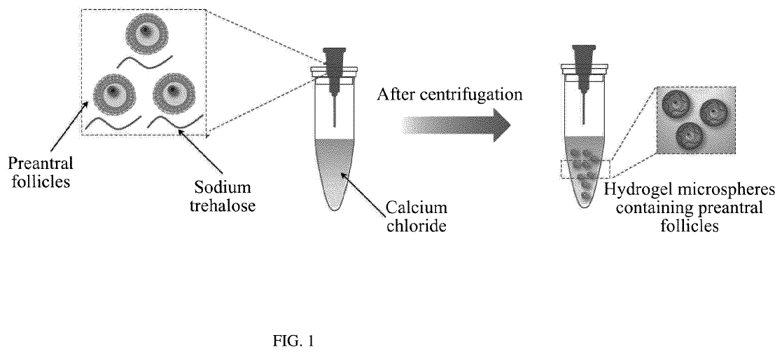

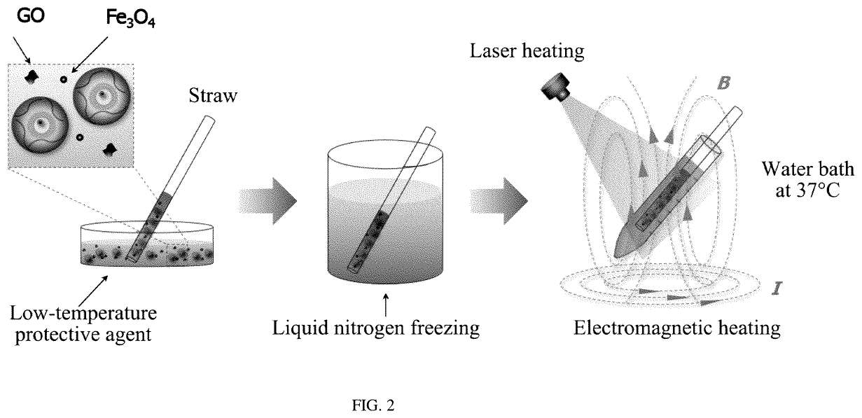

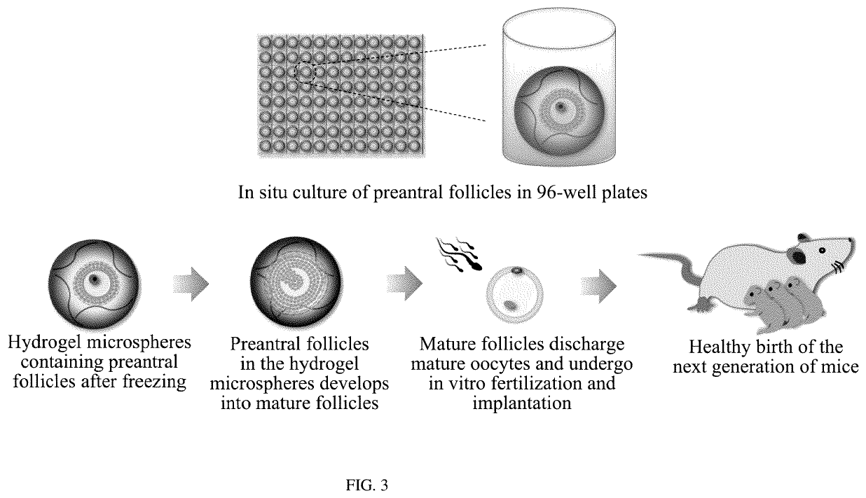

This embodiment combines hydrogel microencapsulation and nano-warming technology to realize the cryopreservation of preantral follicles. To verify the preservation effect, the warmed preantral follicles are further cultured in vitro in three dimensions, including:
1) hydrogel microencapsulation of preantral follicles: a small centrifugal device is used for microencapsulation, where the centrifugal device is made of a 1.5 milliliters (mL) special centrifugal tube and a nozzle; the nozzle (inner diameter of 300 micrometers (μm)) is fixed on the special centrifugal tube through a hole on the tube cover; firstly, 0.3 mole per liter (mol/L) calcium chloride solution is added into the special centrifugal tube, and sodium trehalose solution containing preantral follicles (with a concentration of 1 weight percentage (wt %)) is added into the nozzle, and centrifuged at 100×g for 2 minutes (min) to obtain trehalose gel microcapsules containing preantral follicles, which are then collected into the culture medium for subsequent operation (FIG. 1);
2) incubation with protectant A and addition of nanoparticles: the encapsulated preantral follicles are immersed in protectant A for incubation of 20 min, then moved into another low-temperature protectant B for incubation of 10 min (FIG. 2);
3) vitrification cryopreservation of preantral follicles: the protectant B containing nanoparticles and follicles is added to the straw, and then the straw is directly and rapidly immersed in liquid nitrogen for vitrification cryopreservation (FIG. 2);
4) nano-warming of preantral follicles: a 50 mL centrifugal tube containing 37° C. water is placed into an electromagnetic coil energized with 15 amperes (A) current and aligned with the upper side with a turned on near infrared (NIR) laser emitter, the straw in liquid nitrogen is then quickly taken out and placed under a NIR spot in a water bath at a power of 3 watt per square centimeter (W/cm²) for nano-warming of 2-3 seconds (s) (FIG. 2);
5) in vitro in-situ culture of preantral follicles after warming: after warming, the preantral follicles encapsulated by hydrogel in straw are taken out, directly placed in a 96-well plate which has been filled with 100 mL of culture medium A for in vitro culture, and half of the culture medium is changed every other day until preantral follicles develop, then changed into culture medium B, and the culture is continued for 16-22 hours (h) until mature oocytes are developed (FIG. 3).

Culture medium A includes: α-MEM+10% (v/v) FBS+100 mIU/mL FSH+ITS (1×)+1000 IU/mL LIF+5 μg/mL EGF.

Culture medium B includes: α-MEM+10% (v/v) FBS+100 mIU/mL FSH+ITS (1×)+1000 IU/mL LIF+5 μg/mL EGF+200 mIU/mL LH+2.5 U/mL HCG.

### Comparative Embodiment 1

Comparative embodiment 1 combines hydrogel microencapsulation and conventional 37° C. water bath to realize the cryopreservation of preantral follicles. To verify the preservation effect, the warmed preantral follicles are further cultured in vitro in three dimensions, including:
1) hydrogel microencapsulation of preantral follicles: a small centrifugal device is used for microencapsulation, where the centrifugal device is made of a 1.5 mL special centrifugal tube and a nozzle; the nozzle (inner diameter of 300 μm) is fixed on the special centrifugal tube through a hole on the tube cover; firstly, 0.3 mol/L calcium chloride solution is added into the special centrifugal tube, and sodium trehalose solution containing preantral follicles (with a concentration of 1 wt %) is added into the nozzle, and centrifuged at 100×g for 2 min to obtain trehalose gel microcapsules containing preantral follicles, and then collected into the culture medium for subsequent operation (FIG. 1);
2) incubation with protectants and addition of nanoparticles: the encapsulated preantral follicles are immersed in protectant A for incubation of 20 min, then moved into another low-temperature protectant B for incubation of 10 min (FIG. 2);
3) vitrification cryopreservation of preantral follicles: the protectant B containing nanoparticles and follicles is added to the straw, and then the straw is directly and rapidly immersed in liquid nitrogen for vitrification cryopreservation (FIG. 2);
4) rewarming of preantral follicles: a 50 mL centrifugal tube with 37° C. water is rewarmed for 5-7 s;
5) in vitro in-situ culture of preantral follicles after warming: after warming, the preantral follicles encapsulated by hydrogel in straw are taken out, directly placed in a 96-well plate which has been filled with 100 mL of culture medium A for in vitro culture, and half of the culture medium is changed every other day until preantral follicles develop, then changed into culture medium B, and the culture is continued for 16-22 h until mature oocytes are developed (FIG. 3).

Culture medium A includes: α-MEM+10% (v/v) FBS+100 mIU/mL FSH+ITS (1×)+1000 IU/mL LIF+5 μg/mL EGF.

Culture medium B includes: α-MEM+10% (v/v) FBS+100 mIU/mL FSH+ITS (1×)+1000 IU/mL LIF+5 μg/mL EGF+200 mIU/mL LH+2.5 U/mL HCG.

### Comparative Embodiment 2

Comparative embodiment 2 combines hydrogel microencapsulation and Electromagnetic induced heating (MIH) to realize the cryopreservation of preantral follicles. To verify the preservation effect, the warmed preantral follicles are further cultured in vitro in three dimensions, including:
1) hydrogel microencapsulation of preantral follicles: a small centrifugal device is used for microencapsulation, where the centrifugal device is made of a 1.5 mL special centrifugal tube and a nozzle; the nozzle (inner diameter of 300 μm) is fixed on the special centrifugal tube through a hole on the tube cover; firstly, 0.3 mol/L calcium chloride solution is added into the special centrifugal tube, and sodium trehalose solution containing preantral follicles (with a concentration of 1 wt %) is added into the nozzle, and centrifuged at 100×g for 2 min to obtain trehalose gel microcapsules containing preantral follicles, and then collected into the culture medium for subsequent operation (FIG. 1);
2) incubation with protectants and addition of nanoparticles: the encapsulated preantral follicles are immersed in a low-temperature protectant A for incubation of 20 min, then moved into another low-temperature protectant B for incubation of 10 min (FIG. 2);
3) vitrification cryopreservation of preantral follicles: the protectant B containing nanoparticles and follicles is added to the straw, and then the straw is directly and rapidly immersed in liquid nitrogen for vitrification cryopreservation (FIG. 2);
4) MIH of preantral follicles: a 50 mL centrifugal tube with 37° C. water is placed into an electromagnetic coil with 15 A current for MIH of 3-4 s;
5) in vitro in-situ culture of preantral follicles after warming: after warming, the preantral follicles encapsulated by hydrogel in straw are taken out, directly placed in a 96-well plate which has been filled with 100 mL of culture medium A for in vitro culture, and half of the culture medium is changed every other day until preantral follicles develop, then changed into culture medium B, and the culture is continued for 16-22 h until mature oocytes are developed (FIG. 3).

Culture medium A includes: α-MEM+10% (v/v) FBS+100 mIU/mL FSH+ITS (1×)+1000 IU/mL LIF+5 μg/mL EGF.

Culture medium B includes: α-MEM+10% (v/v) FBS+100 mIU/mL FSH+ITS (1×)+1000 IU/mL LIF+5 μg/mL EGF+200 mIU/mL LH+2.5 U/mL HCG.

### Comparative Embodiment 3

Comparative embodiment 3 combines hydrogel microencapsulation and near-infrared laser-induced heating (LIH) technology to realize the cryopreservation of preantral follicles. To verify the preservation effect, the warmed preantral follicles are further cultured in vitro in three dimensions, including:
1) hydrogel microencapsulation of preantral follicles: a small centrifugal device is used for microencapsulation, where the centrifugal device is made of a 1.5 mL special centrifugal tube and a nozzle; the nozzle (inner diameter of 300 μm) is fixed on the special centrifugal tube through a hole on the tube cover; firstly, 0.3 mol/L calcium chloride solution is added into the special centrifugal tube, and sodium trehalose solution containing preantral follicles (with a concentration of 1 wt %) is added into the nozzle, and centrifuged at 100×g for 2 min to obtain trehalose gel microcapsules containing preantral follicles, and then collected into the culture medium for subsequent operation (FIG. 1);
2) incubation with protectants and addition of nanoparticles: the encapsulated preantral follicles are immersed in a low-temperature protectant A for incubation of 20 min, then moved into another low-temperature protectant B for incubation of 10 min (FIG. 2);
3) vitrification cryopreservation of preantral follicles: the protectant B containing nanoparticles and follicles is added to the straw, and then the straw is directly and rapidly immersed in liquid nitrogen for vitrification cryopreservation (FIG. 2);
4) nano-warming of preantral follicles: a 50 mL centrifugal tube containing 37° C. water is aligned with initiated NIR laser emitter placed above, the straw in liquid nitrogen is then quickly taken out and placed under a NIR spot in a water bath (power of 3 W/cm²) for LIH for 2-3 s;
5) in vitro in-situ culture of preantral follicles after warming: after warming, the preantral follicles encapsulated by hydrogel in straw are taken out, directly placed in a 96-well plate which has been filled with 100 mL of culture medium A for in vitro culture, and half of the culture medium is changed every other day until preantral follicles develop, then changed into culture medium B, and the culture is continued for 16-22 h until mature oocytes are developed (FIG. 3).

Culture medium A includes: α-MEM+10% (v/v) FBS+100 mIU/mL FSH+ITS (1×)+1000 IU/mL LIF+5 μg/mL EGF.

Culture medium B includes: α-MEM+10% (v/v) FBS+100 mIU/mL FSH+ITS (1×)+1000 IU/mL LIF+5 μg/mL EGF+200 mIU/mL LH+2.5 U/mL HCG.

### Comparative Embodiment 4

Comparative embodiment 4 combines hydrogel microencapsulation and nano-warming technology (MIH+LIH) to realize the cryopreservation of preantral follicles. To verify the preservation effect, the warmed preantral follicles are further cultured in vitro in three dimensions, including:
1) hydrogel microencapsulation of preantral follicles: a small centrifugal device is used for microencapsulation, where the centrifugal device is made of a 1.5 mL special centrifugal tube and a nozzle; the nozzle (inner diameter of 300 μm) is fixed on the special centrifugal tube through a hole on the tube cover; firstly, 0.3 mol/L calcium chloride solution is added into the special centrifugal tube, and sodium trehalose solution containing preantral follicles (with a concentration of 3 wt %) is added into the nozzle, and centrifuged at 100×g for 2 min to obtain trehalose gel microcapsules containing preantral follicles, and then collected into the culture medium for subsequent operation (FIG. 1);
2) incubation with protectants and addition of nanoparticles: the encapsulated preantral follicles are immersed in a low-temperature protectant A for incubation of 20 min, then moved into another low-temperature protectant B for incubation of 10 min (FIG. 2);
3) vitrification cryopreservation of preantral follicles: the protectant containing nanoparticles and follicles is added to the straw, and then the straw is directly and rapidly immersed in liquid nitrogen for vitrification cryopreservation (FIG. 2);
4) nano-warming of preantral follicles: a 50 mL centrifugal tube containing 37° C. water is placed into an electromagnetic coil energized with 45 A current and aligned with the upper side with a turned on NIR laser emitter, the straw in liquid nitrogen is then quickly taken out and placed under a NIR spot in a water bath (power of 15 W/cm²) for nano-warming for 2-3 s (FIG. 2);
5) in vitro in-situ culture of preantral follicles after warming: after warming, the preantral follicles encapsulated by hydrogel in straw are taken out, directly placed in a 96-well plate which has been filled with 100 mL of culture medium A for in vitro culture, and half of the culture medium is changed every other day until preantral follicles develop, then changed into culture medium B, and the culture is continued for 16-22 h until mature oocytes are developed (FIG. 3).

Culture medium A includes: α-MEM+10% (v/v) FBS+100 mIU/mL FSH+ITS (1×)+1000 IU/mL LIF+5 μg/mL EGF.

Culture medium B includes: α-MEM+10% (v/v) FBS+100 mIU/mL FSH+ITS (1×)+1000 IU/mL LIF+5 μg/mL EGF+200 mIU/mL LH+2.5 U/mL HCG.

### Comparative Embodiment 5

Comparative embodiment 5 combines hydrogel microencapsulation and nano-warming technology to realize the cryopreservation of preantral follicles. To verify the preservation effect, the warmed preantral follicles are further cultured in vitro in three dimensions, including:
preantral follicles encapsulated by hydrogel are directly placed in a 96-well plate with 100 mL of culture medium for in vitro culture, and half of the culture medium is changed every other day until preantral follicles are developed, and then changed into culture medium B, and the culture is continued for 16-22 h until mature oocytes are developed.

Culture medium A includes: α-MEM+10% (v/v) FBS+100 mIU/mL FSH+ITS (1×)+1000 IU/mL LIF+5 μg/mL EGF.

Culture medium B includes: α-MEM+10% (v/v) FBS+100 mIU/mL FSH+ITS (1×)+1000 IU/mL LIF+5 μg/mL EGF+200 mIU/mL LH+2.5 U/mL HCG.

### Performance Comparison

TABLE 1
Survival rate of preantral follicles after freezing

| | Comparative | Comparative | Comparative | Comparative | Embodiment |
|---|---|---|---|---|---|
| Groups | embodiment 1 | embodiment 2 | embodiment 3 | embodiment 4 | 1 |
| Repetition 1 | 59 | 72 | 76 | 60 | 89 |
| Repetition 2 | 56 | 73 | 73 | 65 | 92 |
| Repetition 3 | 55 | 70 | 72 | 62 | 90 |
| Average | 56.7 | 71.7 | 73.7 | 62.3 | 90.3 |
| value (%) | | | | | |

The results show that, compared to the other treatment groups, the survival rate of preantral follicles after freezing obtained in the Embodiment 1 is the highest, and there is a significant difference compared to each of the other control groups (p<0.05).

TABLE 2
Follicle diameter development after freezing

| Groups | 1 day | 4 days | 7 days | 10 days | 13 days |
|---|---|---|---|---|---|
| Comparative | 125 μm | 250 μm | 295 μm | 356 μm | 362 μm |
| embodiment 5 | | | | | |
| Embodiment 1 | 127 μm | 246 μm | 298 μm | 357 μm | 360 μm |

The results suggest that follicles from Embodiment 1 that underwent cryoretention behave consistently with fresh follicle development that does not undergo cryoretention during culture relative to Comparative embodiment 5, indicating that hydrogel encapsulation and nanoretention technology combining photothermal and magnetothermal heat do not affect the capacity of pre-sinusoidal follicles to develop and grow.

The above represents only preferred embodiments of the present disclosure, and it should be noted that for a person of ordinary skill in the art, a number of improvements and modifications are possible without departing from the principles of the present disclosure, which should also be regarded as the scope of protection of the present disclosure.

What is claimed is:

1. A method for cryopreservation of follicles, comprising encapsulating follicles with sodium trehalose gel, followed by moving into a protective solution A for incubation, then moving into a protective solution B for another incubation, and immersing in liquid nitrogen for cryopreservation;
wherein the encapsulating follicles with sodium trehalose gel comprises: using a small centrifugal device, mixing follicles with 0.5 wt %-2 wt % sodium trehalose solution, with a nozzle of an inner diameter of 340 μm, centrifuging at 100×g for 2 min, and dropping with 0.1 mol/L-0.5 mol/L calcium chloride solution;
the protective solution A comprises water and 1 mol/L of ethylene glycol, 1 mol/L of propylene glycol and 1 mol/L of trehalose; and
the protective solution B comprises water and 1 mol/L ethylene glycol, 1 mol/L propylene glycol, 1 mol/L trehalose, 0.5 wt % Fe₃O₄ nanoparticles and 0.05 wt % graphene oxide nanoparticles.

2. The method for cryopreservation of follicles according to claim 1, wherein the follicles are preantral follicles.

3. A method for resuscitating frozen follicles obtained by the method for cryopreservation of follicles according to claim 1, comprising: placing the frozen follicles at 37° C., and carrying out magnetic induction heating and near infrared heating.

4. The method for resuscitating according to claim 3, wherein the frozen follicles are in a straw, and the placing at 37° C. comprises placing in a water of 37° C.

5. The method for resuscitating according to claim 3, wherein a current of the magnetic induction heating is 5 A to 30 A.

6. The method for resuscitating according to claim 4, wherein a power of the near infrared heating is 1 W/cm² to 10 W/cm².

7. An in vitro culture method for the follicles obtained by the method for resuscitating according to claim 3, comprising: culturing resuscitated cells in a culture medium;
In vitro culture method for obtaining follicles
wherein the culture medium comprises a culture medium A and a culture medium B;
the culture medium A comprises: α-MEM, 8% (v/v)-12% (v/v) FBS, 80-120 mIU/mL FSH, 1×-2×ITS, 800-1200 IU/mL LIF and 3-7 g/mL EGF; and
the culture medium B comprises: α-MEM, 8% (v/v)-12% (v/v) FBS, 80-120 mIU/mL FSH+1×-2×ITS+800-1200 IU/mL LIF+3-7 μg/mL EGF, 150-250 mIU/mL LH and 2-3 U/mL HCG.
